(19) United States
(12) Patent Application Publication
(10) Pub. No.: US 2016/0010045 A1
(43) Pub. Date: Jan. 14, 2016

(54) METHOD OF MANUFACTURING A DEVICE FOR SUPPORTING BIOLOGICAL MATERIAL GROWTH AND DEVICE THEREFROM

(71) Applicants: NANYANG TECHNOLOGICAL UNIVERSITY, Singapore (SG); NATIONAL UNIVERSITY OF SINGAPORE, Singapore (SG)

(72) Inventors: Yehuda COHEN, Singapore (SG); Christopher SHEARWOOD, Singapore (SG); Vishal PRAJAPATI, Singapore (SG)

(21) Appl. No.: 14/771,434

(22) PCT Filed: Feb. 25, 2014
(86) PCT No.: PCT/SG2014/000083

§ 371 Date: Aug. 28, 2015

Related U.S. Application Data
(60) Provisional application No. 61/770,627, filed on Feb. 28, 2013.

(51) Int. Cl. C12M 1/12; B29C 45/00; C12M 1/34

(52) U.S. Cl. CPC C12M 23/04; C12M 41/36; C12M 41/34; B29C 45/006; B29K 2069/00

(57) ABSTRACT

Provided is a method of manufacturing a device for supporting biological material growth, including forming a first platform layer through a molding process, the first base layer including a central chamber; and a plurality of elongate channels coupling the central chamber to the periphery of the first platform layer; forming a second platform layer through a molding process; and coupling the first platform layer to the second platform layer. Accordingly, a device for supporting biological material growth is also provided.

### CROSS-REFERENCE TO RELATED APPLICATIONS

The present application claims the benefit of the U.S. provisional patent application No. 61/770,627, filed on 28 Feb. 2013, the entire contents of which are incorporated herein by reference for all purposes.

### TECHNICAL FIELD

The present disclosure describes embodiments generally relating to a method of manufacturing a device for supporting biological material growth and a device for supporting biological material growth.

### BACKGROUND

The developmental biology of bacterial biofilm is largely driven by diffusion processes and hence it is governed by the dynamics of environmental gradients in their microenvironments. The study of mixed-species biofilm requires the careful establishment of these gradients in the relevant spatial resolution in a dedicated biofilm growth chamber.

Most natural bioprocesses are carried out by mixed-species microbial communities that are organized in compact biofilms, as coordinated communal metabolism requires close physical proximity and aggregation. The power of these complex communities in driving key bioprocesses has been demonstrated in many systems, such as seen by the ability of natural cyanobacteial mats to effectively degrade crude oil spills in coastal environments; the efficient degradation of polycyclic hydrocarbons by granulated microbial consortia; and the effective removal of excess combined nitrogen by biofilters in intensive aquaculture. While these bioprocesses are clearly the result of communal metabolism of complex microbial communities, the means by which the community members are organized and interact to enable the overall chemical transformations are not understood, and hence to date, only empirical engineering practices have been used in attempts to optimize the desired bioprocess.

The main limitations in understanding, exploring and controlling the communal activities of microbial biofilms are the lack of tools for reproducible mixed culture experimentation that are essential to set the foundation for future biofilm engineering.

Presently, well-controlled microbiological laboratory experiments are largely limited to the study of single axenic cultures. Moreover, the study of interactions of different microbial species has mostly been carried out in homogeneous mixtures of bacterial cultures in continuous cultures setups.

FIG. 1A illustrates a currently available fluid chamber device. The device 10 includes elongate one-directional chambers in which fluid is injected in at one end and drained out at an opposite end. In FIG. 1A, the device is machined from a Perspex sheet, sandwiched between two aluminum face plates whose function is to hold glass slides. Water tightness is provided by the use of silicone gaskets in between the glass slide and the Perspex. The entire arrangement is held together by screws, and can be disassembled, cleaned and reused. Fluid flow through the observation chamber is essentially one dimensional.

FIG. 1B illustrates a currently available two-dimensional fluid chamber device. A 2-dimensional planar flow cell 12 is provided which supports biofilm growth under 2-dimensional fluid flow conditions. Control of flow conditions is used to create well-defined physical and chemical gradients, which affects biofilm heterogeneity. The planar flow cell is a non-disposable device, constructed from two pieces (10 cm2) of acrylic plastic separated by a 0.6 mm thick sheet of silicone rubber. A square aperture (3.5 cm2) in the centre of the rubber defined the chamber of the flow cell. Stainless steel tubing with I.D.¼0.24 mm and O.D.¼0.50 mm is inserted into the silicone rubber sheet to provide connections for flow inlets and outlets. A glass microscope coverslip is placed over the chamber to enable observation by microscopy with a rubber mask placed on top of the coverslip to protect the glass. Twelve screws around the perimeter of the flow cell are used to seal the system, which must be water-tight to prevent leaks and maintain sterility during long-term operation.

The morphology of biofilms is generally examined using high resolution confocal microscopy restricting the separation between the microscopes objective lens and the plane of the biofilm to be less than 1 mm. This requires the surface of fluid chamber devices to be flat and the glass slide to be thin so that this objective can be achieved. In addition, physical sampling of biofilms during growth is often required and so easy access is important. It is noted that both devices described above do not satisfy these criteria.

Micro-sensing tools in a variety of natural microbial mats which have been developed help to demonstrate that microbial interactions in complex communities are governed by micro-environmental gradients and a spatially heterogeneous assemblage of micro-niches. Hence, in order to harness these bioprocesses, it is desired to develop robust reproducible biofilm growth chambers to carefully examine the importance of environmental micro-heterogeneity for biofilm performance. Such systems represent the future foundation for engineering mixed culture bioreactors designed for specific bioprocesses.

### SUMMARY

According to various embodiments in the present disclosure, there is provided a method of manufacturing a device for supporting biological material growth, including forming a first platform layer through a molding process, the first platform layer including a central chamber; and a plurality of elongate channels coupling the central chamber to the periphery of the first platform layer; forming a second platform layer through a molding process; and coupling the first platform layer to the second platform layer.

According to various embodiments in the present disclosure, there is provided a device for supporting biological material growth, including a first platform layer, formed through a molding process, the first platform layer including a central chamber; and a plurality of elongate channels coupling the central chamber to the periphery of the first platform layer; and a second platform layer, formed through a molding process; wherein the first platform layer is coupled to the second platform layer to form a growth platform.

### BRIEF DESCRIPTION OF THE DRAWINGS

In the drawings, like reference characters generally refer to the same parts throughout the different views. The drawings are not necessarily to scale, with emphasis instead generally being placed upon illustrating the principles of the present disclosure. It is to be noted that the accompanying drawings illustrate only examples of embodiments of this disclosure and are therefore not to be considered limiting of its scope, for the disclosure may admit to other equally effective embodiments. In the following description, various embodiments of the disclosure are described with reference to the following drawings, in which:

FIG. 1A illustrates a currently available fluid chamber device.

FIG. 1B illustrates a currently available two-dimensional fluid chamber device.

FIG. 2 illustrates components of a platform according to an embodiment.

FIG. 3A illustrates the attachment of the removable cover to the device body.

FIG. 3B illustrates components of the removable cover for maintaining a seal.

FIG. 4 illustrates passage of fluid flow in the device according to various embodiments.

FIG. 5 illustrates a plot referring to biofilm thickness.

FIG. 6 illustrates a component of a device with simple straight channels according to an embodiment.

FIG. 7 illustrates the geometry of a fluidic simulation model used according to an embodiment.

FIG. 8A illustrates a simulation of the 3-dimensional flow fields in the 0° orientation.

FIG. 8B illustrates a simulation of the 3-dimensional flow fields in the 90° orientation.

FIG. 9 illustrates a simulation of the 3-dimensional shear-rate in the 90° orientation.

FIGS. 10A-C illustrates 3-dimensional simulations of mixing patterns of a device according to an embodiment.

FIG. 11 illustrates an experimental configuration according to an embodiment.

FIG. 12A shows an image of the prototype according to an embodiment; used for fluidic verification

FIG. 12B shows the coverslip of FIG. 12A with experimental measurement cells indexed.

FIG. 13 illustrates variance of velocity within a flow cell according to an embodiment.

FIG. 14 illustrates a plot of experimental particle velocity measurements against diagonal distance.

FIG. 15 illustrates a comparison between experimental particle velocity measurements and fluidic simulations.

FIG. 16A illustrates components of a device according to an embodiment of the present disclosure.

FIG. 16B illustrates assembled components of the device of FIG. 16A.

FIG. 17A illustrates a first mixing pattern with oxygen and nitrogen saturated water.

FIG. 17B illustrates a second mixing pattern with oxygen and nitrogen saturated water.

FIG. 18 illustrates a block schematic of a method of manufacturing a device according to an embodiment of the present disclosure.

FIG. 19 illustrates a block schematic of a device according to an embodiment of the present disclosure.

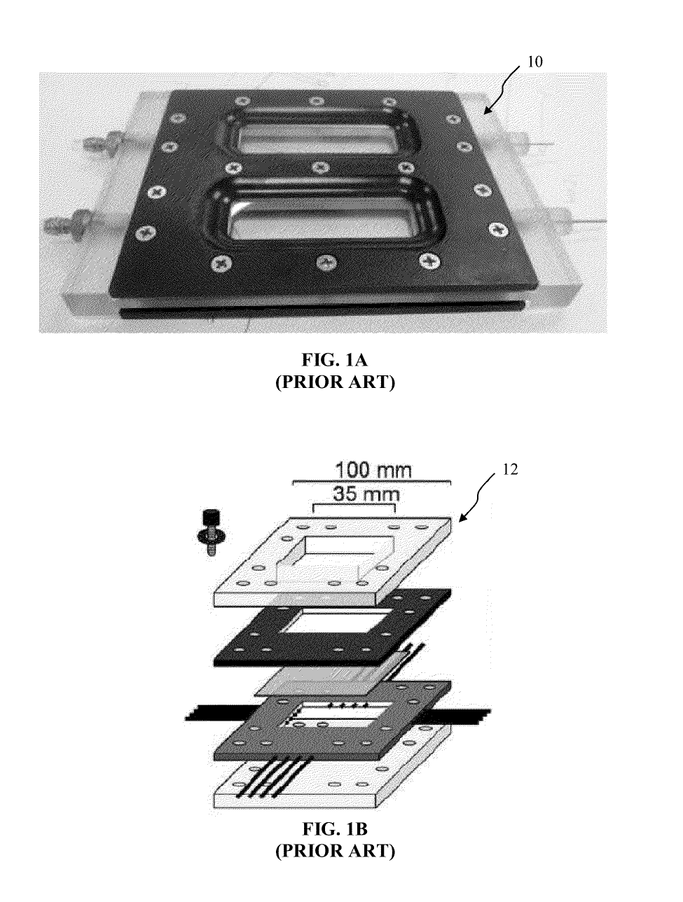

### DETAILED DESCRIPTION

Embodiments of a method of manufacturing a device for supporting biological material growth and a device for supporting biological material growth are described in detail below with reference to the accompanying figures. However, it should be understood that the disclosure is not limited to specific described embodiments. It will be appreciated that the embodiments described below can be modified in various aspects, features, and elements, without changing the essence of the disclosure. Further, any reference to various embodiments shall not be construed as a generalization of any inventive subject matter disclosed herein and shall not be considered to be an element or limitation of the appended claims except where explicitly recited in a claim(s).

According to various embodiments, depiction of a given element or consideration or use of a particular element number in a particular FIG. or a reference thereto in corresponding descriptive material can encompass the same, an equivalent, or an analogous element or element number identified in another FIG. or descriptive material associated therewith. The use of “I” herein means “and/or” unless specifically indicated otherwise.

The present disclosure can describe embodiments of a system or apparatus which can be operable in various orientations, and it thus should be understood that any of the terms “top”, “bottom”, “base”, “down”, “sideways”, “downwards” etc., when used in the following description are used for convenience and to aid understanding of relative positions or directions, and not intended to limit the orientation of a system or apparatus. It is also noted that the term “distal” is used to indicate a location or a portion situated away from a point of origin and the term “proximal” is used to indicate a location or a portion situated toward the point of origin.

According to various embodiments, there is provided a method of manufacturing a device for supporting biological material growth, including: forming a first platform layer through a molding process, the first base layer including: a central chamber; and a plurality of elongate channels coupling the central chamber to the periphery of the first platform layer; forming a second platform layer through a molding process; and coupling the first platform layer to the second platform layer.

According to various embodiments, the present platform for biofilm growth has the potential to enable reproducible studies on thin biofilms as they are grown on different substrates under controlled heterogeneous environmental conditions. Such a device, or platform, or flow cell, has been carefully optimized to be able to establish defined physical-chemical gradients. The device sets the stage for the study of mixed species biofilms in the relevant spatial and temporal resolution.

In embodiments, the device allows for the study of various open questions in biofilm development, for example, the settlement of bacteria from the planktonic state to the formation of thin biofilms; the reverse dispersal processes involved in the transformation of biofilms to planktonic mode; the surface characteristics affecting the kinetics of biofilm formation; inter-species interactions during biofilm development and prey-predator relations in biofilm setting.

This device provides reproducible physical gradients such as control of flow velocity in three dimensions and as such can also control the shear rate gradients on the surface of the device affecting the initial steps of bacterial attachment in a reproducible manner.

In addition to physical gradients, this device has the potential of creating defined chemical gradients with the choice of having more than one media inflow in the chamber under various configurations. In doing so, it is possible to reproducibly grow biofilms under defined heterogeneous environmental gradients and assess the role of these heterogeneous gradients in the overall bioprocesses of the biofilm with greater details. Accordingly, in various embodiments, software or a mobile app is provided for paired operation with this device, so that biologists can easily determine the fluidic characteristics at any point within the device and correlate this with a variety of experimental approaches for the study of mixed-species biofilms behavior. These characteristics include flow velocities, shear forces, etc.

The chemical gradients are correlated with the mixing pattern within the chamber before there is any significant bacterial activity. A series of events of bacterial attachment on the surface will take place depending upon the pre-established chemical gradients as well as the physical setting. Thereafter the changes in chemical gradients created as a result bacterial uptake can be calculated using dedicated software.

In embodiments, the fluidic device has been designed to be an experimental platform for microscopic real time observation of biofilm development. In embodiments, the device has been optimized for the use of standard microscopy borosilicate glass coverslips with standard thickness 0.17 mm and dimensions 22×22 mm.

In embodiments, certain capabilities are provided for bubble removal. However, the prevention of upstream contamination within the device from biological material growth can be controlled by technologies such as Flow Breakers and UV light sources.

In various embodiments, a robust examination of the hydrodynamics or fluidics of the design through simulation together with experimental validation is provided. In embodiments, the manufacturability of the device is also considered and provided, and seeks to meet a criteria of being disposable as a low unit cost, and as such a viable tool to the biological community. Further, in various embodiments, a feature of selective fouling is provided for the device.

In providing a fluidic device as described in various embodiments, it is sought to establish novel biofilm engineering that capitalizes on the precise means by which the different members of the biofilm engineering community interact, including that of communication signals and co-metabolism, as controlled by micro-environmental gradients in the biofilm.

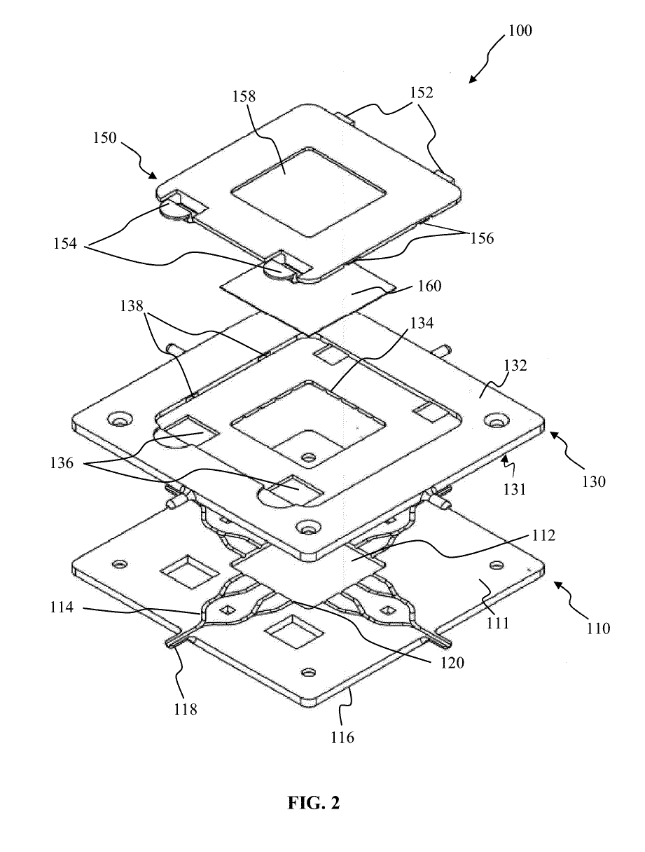

FIG. 2 illustrates components of a platform according to an embodiment. Platform 100 includes a number of separate components. According to an embodiment, a first base layer 110 is provided, and arranged to cooperate with a second base layer 130. A combination or a coupling of the first base layer 110 and the second base layer 130 forms a device body or a growth platform. The device body is designed to have equal and perpendicular sides, in the shape of a square, to allow ease of control of fluid flow and direction. In other embodiments, other shapes can be used, as long as the in and out-flow of fluid is favorably supported.

According to various embodiments, the device body, formed by the first base layer 110 and the second base layer 130, is manufactured by a molding process. In embodiments, the first base layer 110 is formed through a first molding process, and the second base layer 130 is formed through a second molding process. In embodiments, the first molding process is the same as the second molding process. Plastic injection molding is utilized in embodiments as a preferred mass manufacturing method which is suited to polymeric structures of a shell-like design. Advantageously, such a method helps toward the device being a disposable device with a low per-unit cost. In addition, bacteria are very sensitive to metals, so a device fabricated according to various embodiments can avoid negative consequences of exposing the bacteria to metals. Further, there is a limited solubility of metal into solution, which can, in principle, affect the development downstream of bacteria.

In embodiments, the first base layer 130 is formed through plastic injection molding, in which a liquid polymer is injected into a hollowed-out block or mold. The liquid hardens out in the mold and adopts the shape of the mold. A top face 111 of the first base layer 110 is provided with various features provided for the function of the growth platform and formed during the molding process. The bottom face of the first base layer 110 is provided to be generally flat and devoid of features and for resting the growth platform on. In various embodiments, a central chamber 112 is provided in first base layer 110, in which is designed for the support and growth of biological material, such as biofilm. Biofilms can be understood to be a group of microorganisms in which cells stick to each other on a surface.

The central chamber 112 is arranged to be provided in the center of the first base layer 110. In the embodiment, the central chamber 112 is provided geometrically central in the first base layer 110. In an embodiment, the sides of the first base layer 110 and the growth platform is provided at 60 mm, and the sides of the central chamber 112 is provided at 20 mm. The chamber size can also be varied from millimeters in size to being meters in size, depending on the desired outcome. For example, if by the application of specific micro-hydraulic gradients, mixed heterogeneous biofilms can be grown, then this might require a chamber that is meters rather than cm in dimensions. The minimum size, on the other hand, may be determined by the diameters of the input/output channels and the requirements of throughput or laminar flow or quantity of biomass.

A plurality of channels 114 is provided to connect the central chamber 112 to the external edge or the periphery 116 of the first base layer 110. In embodiments, the channels 114 are coupled to a peripheral aperture which is an extruded aperture 118 formed on the periphery 116 of the first base layer 110. The extruded aperture 118 allows for easy connection to a mechanically-operated fluid flow delivery system, which allows for fluidic conductance to and from the central chamber 112, so as to support biological material growth. The extruded aperture 118 is designed to support a single channel within the extrusion, the channel for coupling to a corresponding fluid delivery carrier in a flow delivery system. In various embodiments, the extrusion is left out and the peripheral aperture is provided on the device periphery 116 and within the device body. In such cases, the peripheral aperture can be of an enlarged dimension, such as to receive a plug insert for a mechanically operated fluid flow delivery system. The peripheral aperture can also simply be of a dimension similar to the dimension of the channels. This allows easy coupling to a corresponding growth platform in joint experimental growth sequences.

According to various embodiments, the channel in the extruded aperture 118 is designed to carry fluid to and from the extruded aperture 118 to the central chamber 112. For convenience and efficiency, embodiments in the present disclosure provide for a branch arrangement of channels 114 in the first base layer 110, the channels formed through a molding process. For efficiency, fluid is designed to enter or exit the chamber through four equally spaced channels 120 on one side of central chamber 112, the channels 120 provided in a raised wall of the first base layer 110 surrounding the chamber 112, the wall raised relative to the depressed chamber 112.

In embodiments, the branch arrangement of channels is arranged such that the channel leading in and from the extruded aperture 118 branches out to form the four channels 120 leading to the central chamber 112. In embodiments, the channel leading in and from the extruded aperture 118 is branched twice to form the four channels 120 leading to the central chamber 112. Further, multiple branch arrangements of channels, in this case four, are provided in the first base layer 110, one for each side of the central chamber 112, connecting the central chamber 112 on all sides to the periphery of the first base layer 110, and to a fluid flow delivery system. In embodiments, the branch arrangement includes any number of channels coupling the central chamber to the periphery, and the branch arrangement includes three or four or five branch junctures, or whichever number of channels of branch junctures which satisfactorily supports the growth platform device.

The first base layer 110 is arranged to receive the second base layer 130. In embodiments, second base layer 130 is overlaid over the first base layer 110. In various embodiments, the second base layer 130 is formed through a molding process, and a bottom face 131 of the second base layer 130 is arranged to be overlaid opposingly to the top face 111 of the first base layer 110. In embodiments, the bottom face 131 of the second base layer 130 are further formed with features which are correspondingly similar to those as formed on the top face 111 of the first base layer 110. In coupling both the first base layer 110 and the second base layer 130 together, the features are made complete. In various embodiments, the channel and chamber features of the device 100 can be provided only in the first base layer 110, while the bottom face of the second base layer 130 can simply be flat and smooth.

In embodiments, a semi-circular portion of the channels 114 are formed on the top surface 111 of the first base layer 110. Correspondingly, a semi-circular portion of the channels are also formed on the bottom surface 131 of the second base layer 130. When the base layers are adjoined, the channels 114 are thereby wholly formed. In embodiments, the channels are about 0.8 mm in diameter. In various embodiments, the channels can be of a rectangular shape or any other shape, instead of being round.

With regard to the central chamber 112, a cavity is formed in the first base layer, the cavity not formed fully through the first base layer, but the first base layer still providing a chamber floor to the central chamber. Correspondingly, a through cavity 134 is provided in the second base layer 130, such that in arrangement with the first base layer 110, the central chamber 112 is open from the top surface 132 of the growth platform. In embodiments, a shelf is provided on the perimeter of the chamber cavity 134, and arranged to receive a glass cover-slip, for observation of biological growth activity in the central chamber 112. Other features can also be provided on the top face 111 of the first base layer 110 and the bottom face 131 of the second base layer 130 such that proper alignment of the base layers are validated prior to a permanent sealing of the base layers together.

Attachment or coupling of top face 111 of the first base layer 110 to the bottom face 131 of the second base layer 130 can be provided by a number of mature schemes. For example, any one of a chemical welding, an ultrasonic welding, a laser welding, an indium welding, or adhesion with a bonding adhesive. According to various embodiments, fusion welding is utilized as a method of adhesion, as it takes the two mating surfaces, renders them liquid or tacky, and with pressure creates a monolithic structure, in most cases with no visible seam. In further embodiments, a chemical welding process is used, in which the two mating surfaces 111, 131 are treated with a vapor of chloroform, and thereafter applying pressure to cause the coupling. Such a process can achieve a very high bond strength between the two layers.

The device body can also be fabricated by other manufacturing techniques, for example, with embossing, as well as alternative material types. For example, in a parallel process, a similar device is being fabricated using ceramic glasses and standard microelectronic fabrication techniques. The device body can also be metallic and fabricated by powder injection molding, cast or machined.

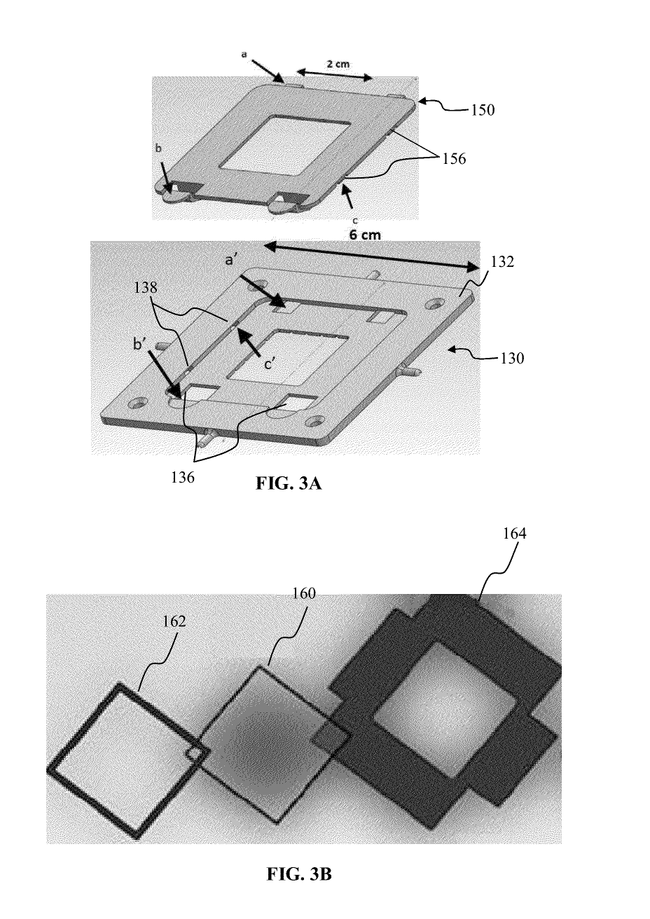

In various embodiments, a removable cover 150 is provided. FIG. 3A illustrates the attachment of the removable cover to the device body. According to embodiments, a function of the removable cover 150 is to locate a glass cover slip 160 and confine it laterally. The device 100 is arranged to be air tight and leak proof with the inclusion of elastomeric gaskets placed between the cover 150 and glass cover slip 160, and the second base layer 130 and the glass cover slip 160.

In embodiments, there are a series of projections 152 located around the edge of the cover. In this case, the tabs 152 are spaced 2 cm from each other. In fitting the cover 150 into the device body, the cover 150 is inclined, and the two projections 152 at position a are made to fit into the corresponding recesses a′ located within the top portion of the body, on the top face 132 of second base layer 130. When inserted, the projections 152 lock into the device body and provide a fulcrum action. As the cover 150 is pressed down, resilient clips 154 at b are co-located with the socket features 136 at b′ and lock the cover into place. By lifting the clips 154 at b, the cover 150 can be removed. To improve the stability of the cover 150, there are provided four tabs 156 in the base of the removable cover 150, at c (two tabs on each side). The tabs 156 can be pushed into their corresponding mating features 138 on the top face 132 of the device body at c′. When the projections 152, the resilient clips 154 and the tabs 156 are received in their complimentary features on the body at a′, b′, and c′ respectively, the cover becomes locked in place.

The cover 150 is also manufactured by plastic injection molding, from polycarbonate, or any thermoplastic polymer. In embodiments, the polymer type for the removable cover 150 can be different from that of the device body, and is mainly determined by the elastic modulus and thickness of the sealing gasket. In embodiments, the removable cover 150 can be made of metal, for example, such as aluminum, formed using traditional sheet-metal processing techniques. The bacteria are never exposed to the cover.

In various embodiments, to prevent damage to the fragile glass cover-slip 160, a protection gasket 162 fabricated from an elastomeric material, is used. FIG. 3B illustrates components of the removable cover for maintaining a seal. A protective gasket 162 is placed over the glass cover-slip 160 and positioned within the aperture 158 of the removable cover 150. An adhesive elastomer can be used to adhere the gasket 162 to the cover 150. To provide the device as fluid tight, a sealing gasket 164 is placed on the shelf 134 formed on the second base layer 130, for receiving the glass cover-slip 160. The sealing gasket 164 is first placed on and around the shelf 134 on the top face 132 prior to receiving the glass cover-slip 160. The removable cover 150, with the glass cover-slip 160 and protective gasket 162 in place, is thereafter locked into position on the top face 132 of the device body 100.

The thickness of the elastomer depends upon its elastic modulus and the material chosen to manufacture the removable cover 150. If the cover 150 is made from polycarbonate, an elastomer made of silicone with a thickness of 100 microns is suitable. Once the cover 150 is pressed into position it will compress both the protective gasket 162 and sealing gasket 164 so that the cover-slip 160 is located both laterally and vertically, and sealing is achieved. In an embodiment, gaskets made from low gas permeability materials, for example, such as neoprene, can also be used.

In various embodiments, a glass cover-slip is used. However, other materials can be used depending upon the type of microscopy used to monitor the biofilm's development. Alternatives include, but are not limited to, quartz, diamond and certain transparent polymers. Both quartz and diamond are extremely hard and strong, allowing a reduction in the thickness of the glass cover-slip 160 whilst still maintaining rigidity. Also, their background fluorescent signal is lower than that of sodium glass.

In embodiments, a glass cover-slip is provided on a top side and a bottom side of the device body. In such a case, the first base layer is formed with a cavity all the way through the base layer. A glass cover slip can there be securely coupled into the cavity for the purposes of observation through the bother side of the combined device body. According to embodiments, cover-slips can also be interchanged with a wide range of coupons to evaluate material sensitivity to biofilm fouling for areas such as for example, medical, membrane, and maritime designs industries.

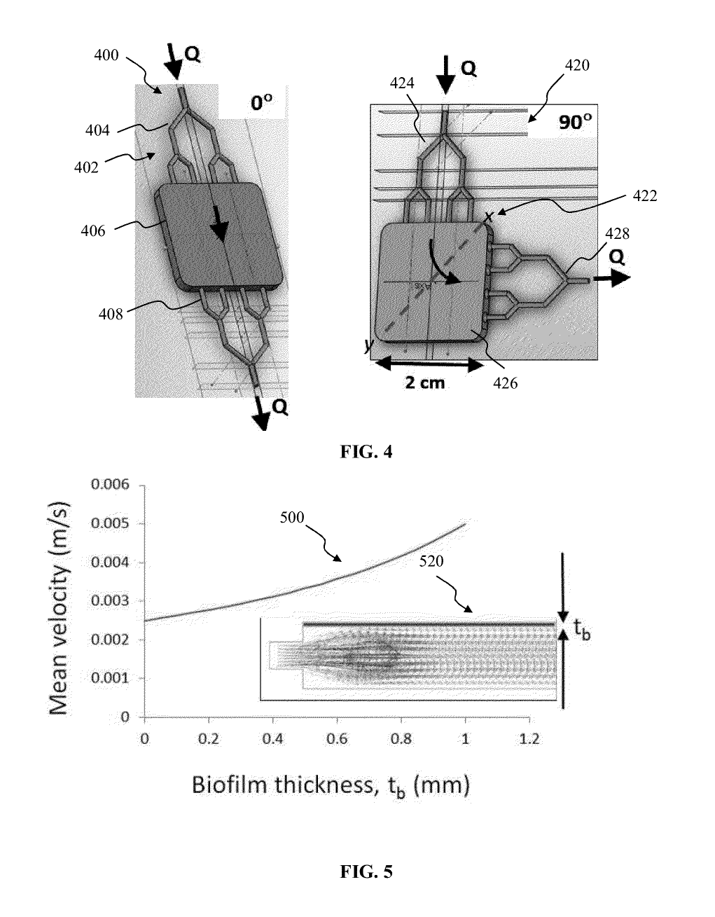

FIG. 4 illustrates passage of fluid flow in the device according to various embodiments. 400 shows a device 402 in which fluid is passed through a first branch arrangement of channels 404, and into a central chamber 406. The fluid is then directed out the chamber 406 through a second branch arrangement of channels 408, where the first and the second branch arrangement of channels each include a general direction of flow through the channels, the angle between the direction of flow of the first branch arrangement and the direction of flow of the second branch arrangement being about 0°. In such a case, the fluid enters and exits the chamber unidirectionally or straight through the device.

420 shows a device 422 in which fluid is passed through a first branch arrangement of channels 424, and into a central chamber 426. The fluid is then directed out the chamber 426 through a second branch arrangement of channels 428, where the first and the second branch arrangement of channels each include a general direction of flow through the channels, the angle between the direction of flow of the first branch arrangement and the direction of flow of the second branch arrangement being about 90°. In such a case, the fluid enters and exits the chamber perpendicularly or where the fluid turns perpendicularly prior to leaving the chamber.

The device or growth platform 422 contains a central chamber 426 with internal geometry of volume a²t where a is the square chamber width, which is 2 cm according to an embodiment and t is the chamber thickness, which is 2 mm according to the embodiment. The top portion of the chamber contains a glass cover-slip, for the eventual growth of a biofilm. Fluid enters and exits the chamber through four equally spaced channels of 0.8 mm diameter. The fixed internal geometry of the device present a constant fluidic conductance to a mechanical pump connected to the inlet manifold. As such, the fluid characteristics or hydraulics of the channels and chamber can simple be defined by the throughput, Q, of fluid flowing into the device. The throughput Q is given by:

Q=vA (1)

where v is the fluid velocity and A is the cross-sectional surface area.

It can then be observed that the fluid velocity in the channels or chamber is determined by the relevant cross-sectional area. This assumes that the surface friction factor is negligible and that there are no leaks. Since the cross-sectional area of the channels are significantly smaller than that of the chamber it implies that the fluid velocity in the channels is significantly larger (>20) than within the chamber.

As long as the fluid velocity is low, the Reynold's number (Re) is also low. It is only when Re>>2,320 that the fluid flow is expected to change from laminar to turbulent. According to embodiments, in the system described, Re<5, and so the fluid flow is uniform, smooth and predictable.

In short, for a given throughput, which can be defined by the pump settings, the micro-hydrodynamics within the device are fixed and can be easily described by the use of a look-up table to users who are unfamiliar with fluidic modeling. In addition, all parameters that are dependent on the micro-hydraulics can be described similarly.

With an understanding of these figures and values, delivery of solutes such as oxygen, nutrients or antibiotics can be quantified. In time, as the bacteria settle on the glass cover-slip 160 (referring back to FIG. 2), a biofilm will start to grow with thickness t_{b} in the chamber, which has a thickness t. This will gradually reduce the chamber thickness (t−t_{b}) as time progresses. Unless the film is smooth, its growth would result in a change to the friction factor. In addition, the biofilm will start to reduce the concentration of oxygen and nutrients supplied dissolved in the fluid.

The effect of the combination of increased biofilm thickness and roughness on the micro-hydraulics within the chamber is difficult to predict. A reduction in the chamber thickness from t to (t−t_{b}) will lead to a corresponding increase in chamber fluid velocity. Theoretically, the friction factor is independent of channel roughness in laminar flow because the disturbances caused by surface roughness are quickly damped by viscosity.

FIG. 5 illustrates a plot referring to biofilm thickness. 500 shows a plot of mean chamber velocity, in m/s, against the thickness of a smooth, uniform biofilm t_{b}. 520 shows the biofilm projecting to the volume of the chamber. In various embodiments, as long as the biofilm thickness, t_{b}, remains less than 100 microns, the impact on the micro-hydraulics will be minimal. For greater thicknesses, as long as the surface of the biofilm is smooth, its effect on the micro-hydraulics can be predictable and can be compensated for.

According to various embodiments, a wide variety of commercially available sensors can be provided in the device 100 and used to monitor the concentration of critical supplies to the biofilm, including oxygen. Volumetric oxygen sensors can be located at the input and output, which allow the difference in oxygen to be determined for aerobic bacteria. Specialized plant optrodes can be coated onto the coverslip, prior to the growth of the biofilm, so that a 2-dimensional map of oxygen concentration can be produced for this location. Oxygen reporters can be introduced into the chamber to provide for a localized 3-dimensional map of oxygen concentration to be created for within the biofilm's architecture, which can provide for insight down to the cellular level.

According to various embodiments, electrical contact to the chamber can be provided by the fabrication of planar wires in between the top and bottom layers using mature semiconductor processing techniques. Further, an electrical connection or wires can be formed within the device layers during the molding formation, so as to provide an electrical contact through the device body. Similarly, thermocouples can be made to measure temperature or used as a heat pump. The use of a conducting polymer or a surface coating provides a Faraday cage for the device screening the bacteria from the influences of airborne electromagnetic radiation. This can be useful in establishing a baseline of the developmental biology of bacteria in the absence of electromagnetic radiation.

Discrete devices and sensors can be incorporated into the body of the device either during plastic injection molding or afterwards by the provision of suitable recesses, according to various embodiments. For example, either permanent magnets, for example, and not limited to, neodymium magnets or samarium-cobalt magnets, or other rare-earth magnets, can be placed close to the chamber to provide a static magnetic bias. A magnetometer can then be provided to observe the magnetic waveforms. This is as bacterial developmental biology is thought to be sensitive to magnetic fields and electromagnetic radiation.

In various embodiments, high permeability structures, such as high permeability discs or cylinders, can be implanted within the body of the device, for example, and not limited to amorphous FeSiBC or Permalloy, which is an iron-nickel alloy. With the use of planar or 3-dimensional coils, this can provide a way to expose the bacteria to high field strength electromagnetic perturbation with variable amplitude and frequency, either parallel or perpendicular to the plane of the biofilms. A biofilm community resembles that of an ancient skin and might be the best model for evaluating the interaction between cells and external perturbations.

According to various embodiments, a variation in the number of channels can be provided. In providing multiple channel arrangements in coupling the central chamber to the periphery of the device body, the channel arrangements could be of the same design, or may include variations in channel design. For example, one channel arrangement could include a single straight channel coupling the chamber to the peripheral aperture, while another channel arrangement could include the branched arrangement as described earlier. Further, more than four channels could be provided in the channel arrangements.

In embodiments, a variation in the channel diameter can be provided. The channel diameter can be dimensioned from micrometer size to centimeter size, depending on the biological matter to be grown. In embodiments, a variation in the channel cross-section can be provided. For example, the channel can be cross-sectioned to be any one of a triangular, pyramidal, elliptical, square, rectangular, or any other shaped. In embodiments, a variation in the channel longitudinal shape can be provided. For example, the channel can be a simple tube, or it can be provided as binary manifolds.

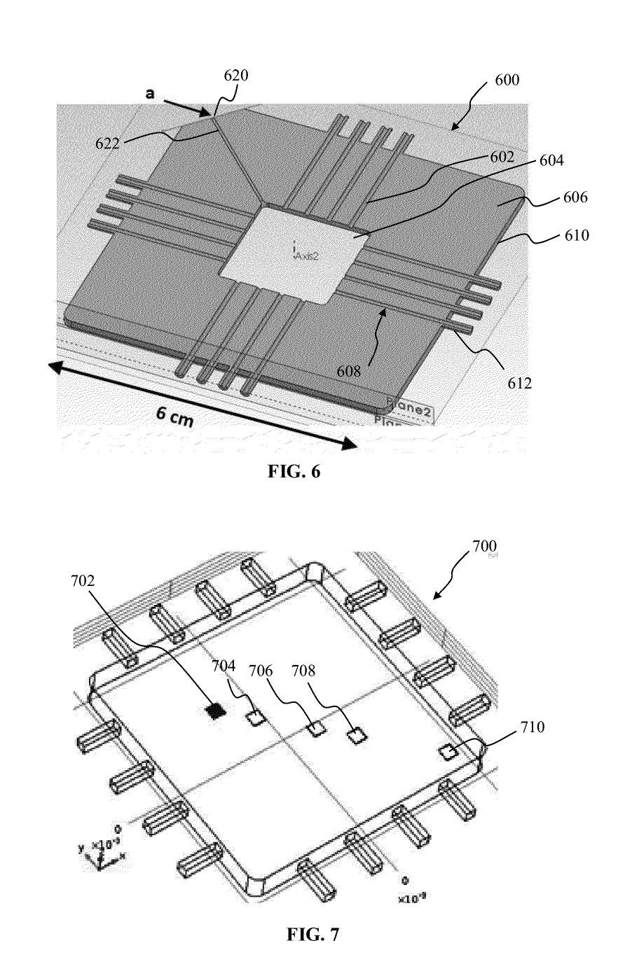

FIG. 6 illustrates a component of a device with simple straight channels according to an embodiment. In device 600, a plurality of channels 602 and a central chamber 604 are formed in base layer 606. Base layer 606 is formed through a molding process and is intended to be coupled with another base layer to form the device body for supporting biofilm growth. In the embodiment, a channel arrangement 608 including four straight channels is provided to couple the central chamber 604 to the periphery 610 of the base layer 606. The four straight channels are directed to four corresponding peripheral extrusion apertures 612 provided on the base layer. In the embodiment, the device is intended to be a square shaped device, each of the sides of the device determined to be 6 cm long.

In embodiments, a service port 620 is provided in the device 600 and located at the position marked “a”. A corresponding service channel 922 is formed in the base layer 606 and connects the central chamber to the port 620. The channel is provided away from the channel arrangements 608 provided for fluid flow. By default, the port 620 and the channel 622 are sealed during manufacture by a thin membrane. The membrane has to be disrupted using a hypodermic needle, and the port 620 can then be used to inoculate bacteria into the device, or to help remove stubborn bubbles during operation. Bubble removal can be facilitated by using a hypodermic syringe and withdrawing the plunger whist connected to this port 620.

The device is meant to operate for periods of hours to months, and permit real time observation of the growth and developmental biology of the biofilms within the chamber, through the glass cover-slip. Consequently, fouling at the inlet/outlets or the extruded apertures 118 should be avoided otherwise the reproducible velocity gradients will start to change in unpredictable ways.

To prevent fouling of the inlet and outlet a number of strategies are possible. First, the high fluid velocity and associated wall shear stress is high within the channels, discouraging settlement and biofilm formation. In addition, micro-embossing can be used to create a texture on the surface of the tubes, discouraging settlement. The choice of polymer also influences the settle of bacteria since this is dependent on the surface energy. In addition, various tailored ceramic coatings can be used to provide local toxicity to the bacteria. These include, but are not limited to, indium tin oxide, zinc oxide, and titanium dioxide. A combination of these schemes can be expected to delay biofilm formation and as such fouling of the extruded apertures for up to a few months. In embodiments, an anti-microbial solution can be provided to inhibit the fouling at the apertures of the device.

In various embodiments, the split design of the device body, in welding a first top device layer and a second bottom device layer together, also allows the coating of metallic or ceramic or polymers layers in between the top and bottom portion. As mentioned previously, these sections can be coupled together using an epoxy resin or cold welded after sputter coating with a layer of indium. This design also permits the localized coating of the channels with metals or polymers or ceramics using mature techniques from the microelectronics industry. For example, metallic template can be machined and mated to either the top or bottom portion. This combination then can be placed in a sputter coating machine allowing any material type, as well as multiple-layers of materials to be locally deposited in selected areas, such as the channels. Then upon assembly of the device, the channels would have an internal coating.

In embodiments, the top part of the device can be unselectively coated with indium metal. In doing so, a consideration is that the process temperature should be less that the glass transition temperature of the polymer that is used to manufacture the body. After coating both the top and bottom portions with indium, the device is treated with dilute hydrochloric acid to remove the oxide layer, and these portions can be mated together to form the assembled device body. In embodiments, a metallic layer sandwiched in between the top and bottom layers can be used as an earth plane.

In embodiments, since the body of the device is transparent, variations in the design could render part or parts of the device into optical waveguides. For example, this might permit a side illumination of the bacteria for purposes of inducing fluorescence or perhaps to simply provide perturbation. A conventional microscope can then be used to measure the response, as compared with having a dual purpose of illumination and response through the same microscope objectives.

According to various embodiments, the design of the growth platform allows for arrays of the chambers to be created by multiple schemes. Individual chambers can be connected together. Alternatively, a monolithic m×n design can be used in which the input and output supplies are multiplexed to the individual elements. This permits rapid assessment of anti-fouling agents and antibiotics, as well as other relevant chemicals, which can include tradition Chinese medicines.

FIG. 7 illustrates the geometry of a fluidic simulation model used according to an embodiment. Model 700 represents a device for supporting biological material growth according to an embodiment. For simplicity, only the entrance and exit channels are used even though the flow cell 700 has a binary input and output network. The channel cross-section of the prototype is square rather than circular due to a manufacturing restriction. It is common practice in such cases to assign an equivalent fluidic diameter to such channels. A series of observational cells 702, 704, 706, 708, 710 are defined along the chamber diagonal to facilitate comparison during subsequent experimental validation. The average velocity under a given flow rate is calculated within the volume of each cell, where the cell depth is defined by the microscope objective's magnification.

After creation of the geometry and defining all the desired parameters to be averaged and calculated with respect to their domains, boundary materials can be selected from the inbuilt material library and as well as the fluid material identified. The boundary material was defined as polycarbonate and the fluid was defined to be Phosphate Buffer Solution (PBS), a buffer frequently used in such experiments. Surface roughness was not taken into consideration as the interior walls are expected to be smooth throughout the operation lifetime of the device. The software used was Comsol Multiphysics, but other fluidic software, for example Ansys Fluent, can also be used.

Boundary conditions were then defined for the inlet and outlets in use. In each orientation, 0° or 90°, only one set of inlet and one set of outlets were used; the redundant ones were sealed off. Laminar flow velocity was defined in all four inlets. The velocity in each inlet was calculated by using equation (1) or v=Q/A. The remaining boundary conditions were defined as the solid wall material.

The flow rate was modeled from near zero to a soft and hard maximum. The soft maximum is defined as the conditions when the flow field experiences some irregularities such as swirling but these irregularities do not interfere with the central area of the flow cell where the biofilm grows and analyzed. The hard maximum corresponds to the point where reproducible gradients over the biofilm are no longer possible. Table 1 gives the soft and hard points that have been determined for this particular geometry.

| Flow Rate (ml/hour) | Comment |
|---|---|
| 150 | Soft limit |
| 250 | Hard limit |

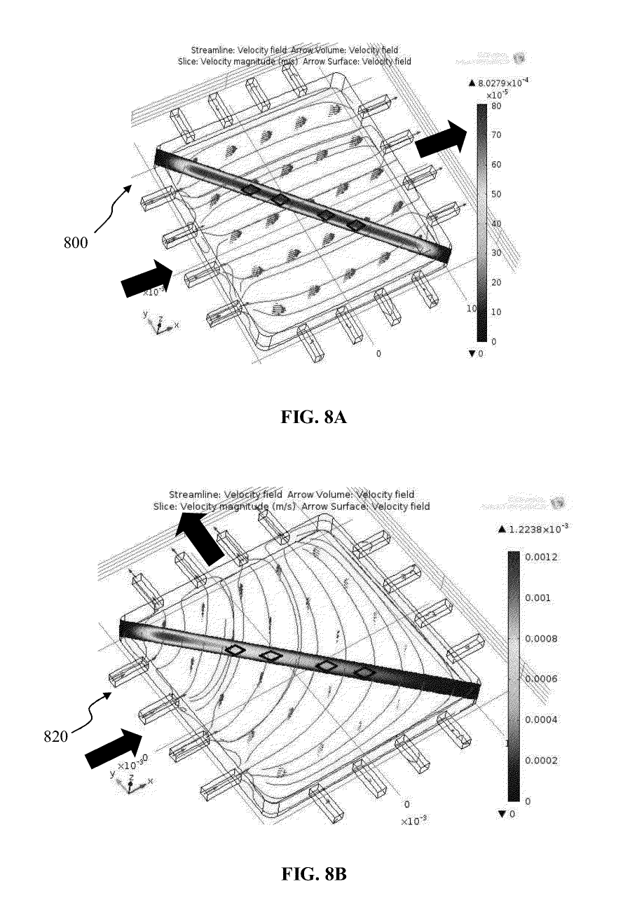

FIG. 8A illustrates a simulation of the 3-dimensional flow fields in the 0° orientation. FIG. 8B illustrates a simulation of the 3-dimensional flow fields in the 90° orientation. 800 shows stream lines across the device in the 0° configuration, while 820 shows stream lines across the device in the 90° configuration. By contrast, in the 90° orientation, there is a velocity gradient created by the different path lengths of the stream lines from nozzle to nozzle. In this example the flow rate was set at 69 ml per hour.

These 3-dimensional velocity fields control the transport of solutes such as oxygen to the boundaries of the biofilm where diffusion takes place. In addition to this the fluid flow produces mechanical forces on the biofilm which, if large enough, can lead to detachment.

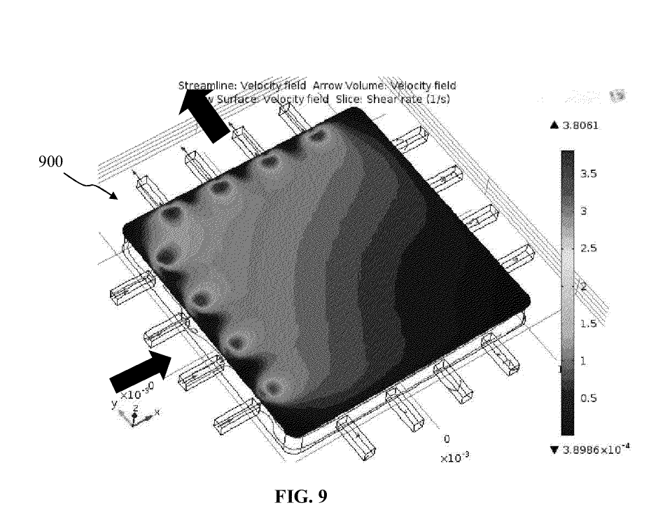

FIG. 9 illustrates a simulation of the 3-dimensional shear-rate in the 90° orientation. 900 shows a 3-dimensional simulation of the shear rate (1/s) of the device in 90° orientation with a flow rate of 69 ml/hr. In 900, the shear rate is plotted in the 2-dimensional plane in which the biofilm will grow. If the shear rate is multiplied by the dynamic viscosity then the shear force is produced. As a consequence of the velocity gradient created by this orientation, a shear rate gradient is also produced. Biofilm developmental biology is dependent on the magnitude of the shear rate.

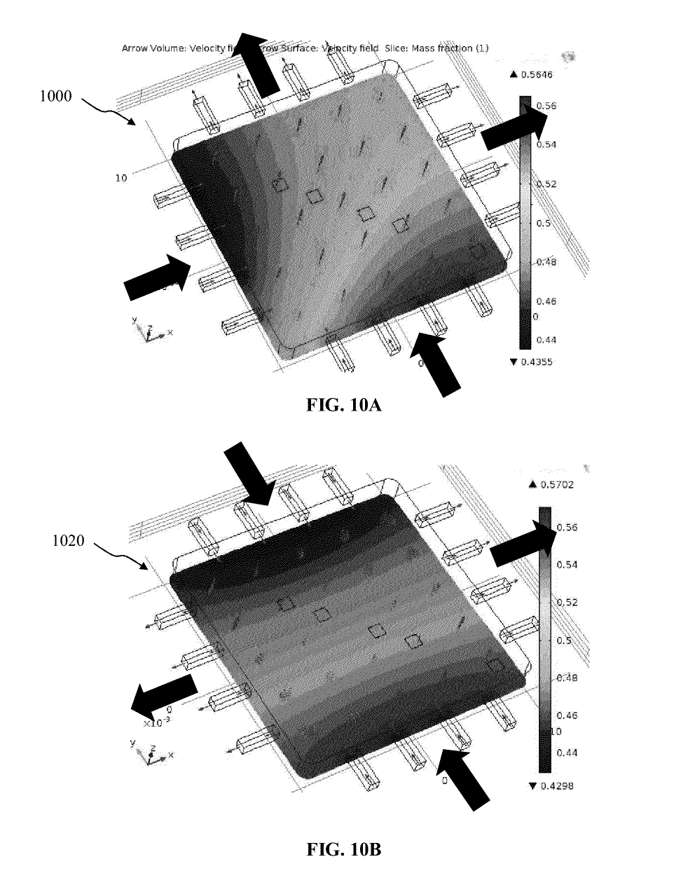

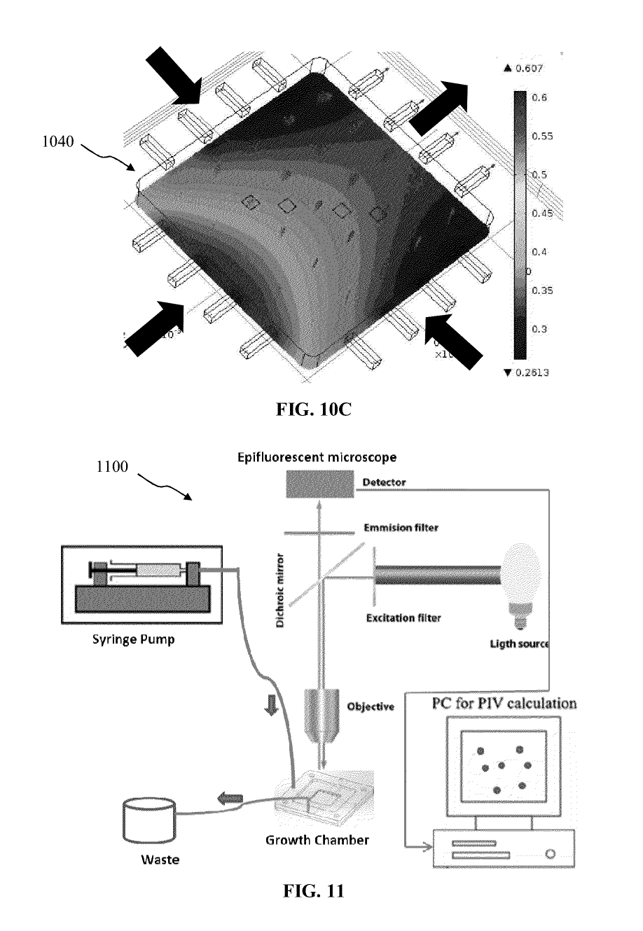

FIGS. 10A-C demonstrate some of the possible mixing patterns in the chamber according to various embodiments, which correlate to initial chemical gradients, and can be realized using this chamber. These chemical gradients created due to each mixing pattern vary extensively with the change in inflow rates. FIG. 10A shows 3-dimensional simulation 1000 where the inflow and outflow of fluids are from adjacent sides. FIG. 10B shows 3-dimensional simulation 1020 where the inflow and outflow of fluids are from opposite sides. FIG. 10C shows 3-dimensional simulation 1040 where the inflow of fluid is provided on 3 sides, while providing one side for outflow.

FIG. 11 illustrates an experimental configuration according to an embodiment. Experimental configuration 1100 is used to validate the fluidic simulations via particle velocity measurement. Particles or in this case 15 μm diameter polystyrene microspheres (FluoSpheres®) are mixed with a standard buffer solution of PBS (Phosphate Buffered Saline) and the concentration adjusted to obtain neutral buoyancy. This solution is loaded into a syringe pump (New Era Pump Systems, Inc. SyringePump.com; Model no: NE-1000) and circulated through the flow cell after suitable priming. All bubbles are carefully removed to enable reproducible flow measurements. After circulating through the flow cell, the solution is collected in a waste bottle.

An Epifluorescent Microscope (Carl Zeiss Axio Imager), with the objective set to 5×, is used to image the mid-plane of the device. Illumination is provided by a Mercury lamp (HXP 120C) and the image is captured by a camera (Carl Zeiss AxioCam MRm) in combination with acquisition software (Zen 2012). Particle image velocity (PIV) calculations are subsequently made on captured video sequences using software (Imaris 7.5). Imaris identifies the particles and tracks them over time-series images to calculation particle motion parameters.

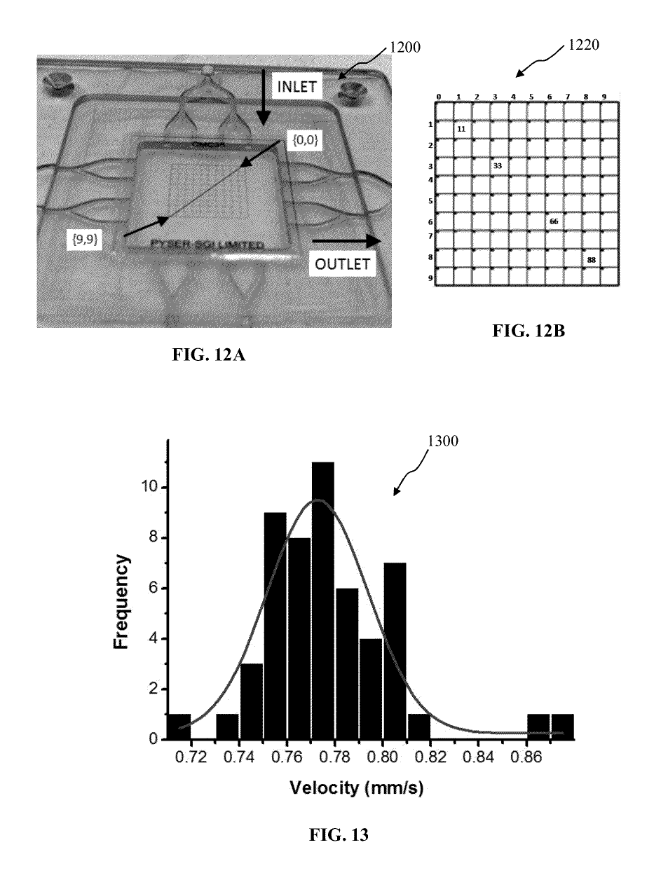

FIG. 12A shows an image of the prototype according to an embodiment. Prototype 1200 is used to validate or verify the fluidic modeling, configured for the 90° mode. The input/outputs not in use are terminated. Instead of a glass cover-slip a graduated plastic coverslip has been used to facilitate measurements and features a 10×10 grid of 1 mm squares, each square individually indexed (Pyser-SGI Limited). The total diagonal length of the grid, corner to corner, is \sqrt{2}×10 mm or 14.14 mm.

FIG. 12B shows the coverslip 1420 of FIG. 12A with experimental measurement cells indexed. Diagonal grids cells {1,1}, {3,3}, {6,6}, and {8,8} were chosen for this study. Across each cell, the particle velocity can be averaged and correlated with the fluidic simulations. With the microscope objective set at 5×, the depth of field was then 36 In essence, even though particles are in focus at a point in the mid-plane of the device, the particle may actually be at any depth with a total uncertainty of 36 μm along the z-axis. Consequently, the particle velocity is really averaged out in a given cell over a box of volume 1 mm by 1 mm by 36 μm. A control software, such as Imaris, identifies the motion of the particles and tracks them over time-series images. Transit times across the diagonal x-y shown in 1500 range from 1.4 to 4.2 seconds.

FIG. 13 illustrates variance of velocity within a flow cell such as {1,1} according to an embodiment. Histogram 1300 shows frequency distribution of the representative data taken in cell {1,1} at a flow rate of 69 ml per hour. A total of 56 data points were collected and analyzed. The distribution is Gaussian in shape and represents random variations around a common mean. A Gaussian fit gives a mean value of 773±3 μm/s with a standard deviation of 42±7 μm/s at a R² of 0.8.

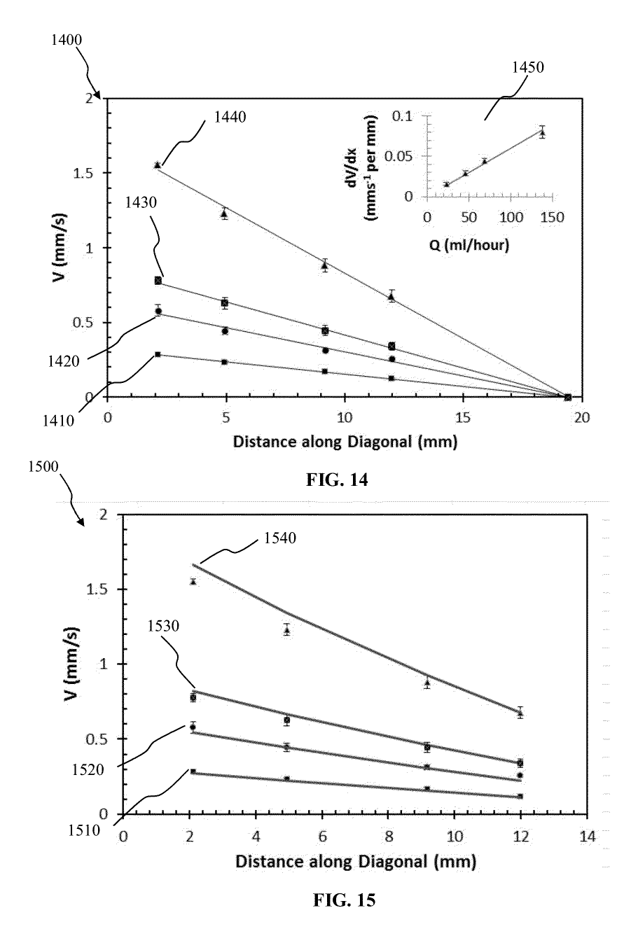

FIG. 14 illustrates a plot of experimental particle velocity measurements against diagonal distance. Plot 1400 shows the experimental data taken for the 90° configuration with flow rates of 23, 46, 69 and 138 ml per hour at the mid-plane of the flow cell. Curves 1410, 1420, 1430, and 1440 are provided for flow rates of 23, 46, 69, and 138 ml per hour respectively. Within experimental limits, regression analysis confirms the trends for the flow rates to be linear. The inset 1450 of FIG. 14 shows the linear dependency of the velocity gradient on flow rate.

FIG. 15 illustrates a velocity plot against diagonal distance. Plot 1500 gives a comparison of the experimental particle velocity measurements to the results from the fluidic simulations, using Comsol software. Curves 1510, 1520, 1530, and 1540 are provided for flow rates of 23, 46, 69, and 138 ml per hour respectively. The close agreement between simulations and experimental data confirmed the validity of the model. Although the agreement is satisfactory these is a non-linear component clearly present in the simulation results. This is believed to be due to neglecting chamber specific details such as material type and surface roughness, which have only a minor impact on the fluidic behavior at this scale length, dominated by laminar flow, with low Reynolds numbers.

Experimental particle velocity measurements in the 0° mode confirm the validity of the fluidic simulations.

According to embodiments, it is an intention to provide a device that is disposable and of low unit cost. Consequently the device has been designed for manufacturability; plastic injection molding being the appropriate technology as it accounts for almost 30% of the world's plastic products. Thin shelled devices are routinely fabricated by plastic injection molding in extremely larger volume. The unique advantage of this technology is the ability to create the entire geometry in one cycle or shot as well as the versatility in changing the plastic from which the device is made. Particular challenges include minimizing defects, controlled stress and surface roughness, and maintaining flatness. Fortunately most of these defects can be eliminated by careful design and optimization of machine parameters such as timing, temperature, and pressure.

According to an embodiment, the main components of the device, first base layer 110, second base layer 130, and removable cover 150, have all been optimized for production by plastic injection molding. Techniques include the use of polymer melt flow simulation software such as Moldflow and the application of design-of-experiment techniques such as the Taguchi Method. Critical parameters requiring special attention include the flatness and smoothness of the mating surfaces (111 and 131). Bonding of the first and second base layers together is a work in progress. Initial results using ultrasonic and laser bonding both appear very encouraging.

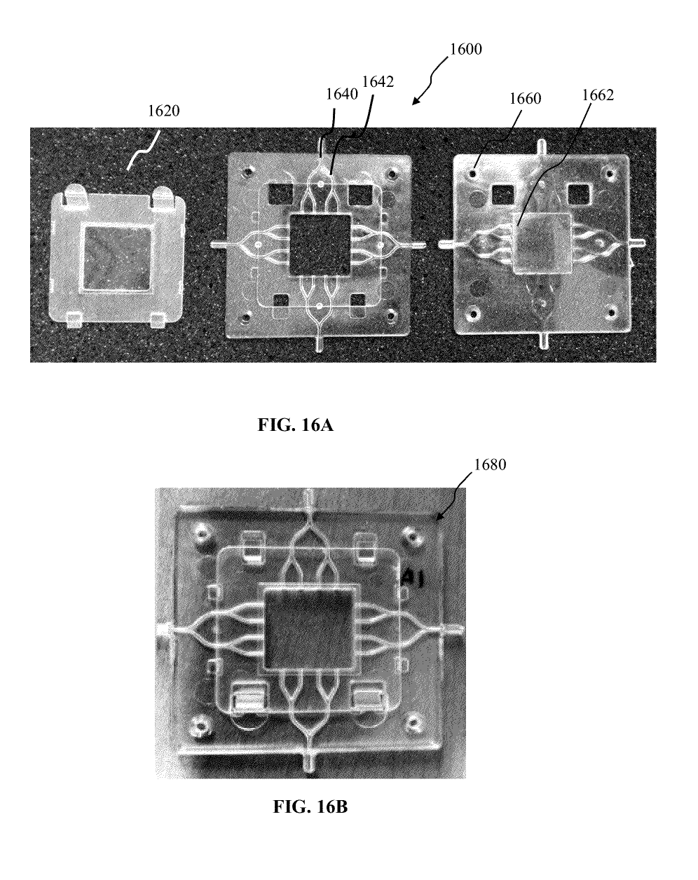

FIG. 16A illustrates components of a device according to embodiments, as manufactured by plastic injection molding. 1620 is the removable cover; 1640 is the second base layer; 1660 is the first base layer. In embodiments, optimization of the plastic injection molding cycle can contribute to the elimination of device-specific defects. According to various embodiments, the outer diameter of channels 1642 should be sufficiently large to avoid hesitation, and the first base layer 1660 should be sufficiently thickened to avoid polymer flow induced cracking of the central chamber 1662. According to an embodiment the outer diameter of the channels 1642 is greater than 2.6 mm.

FIG. 16B illustrates the assembled components of the device of FIG. 16A. Detailed analysis of injection molding based on the Taguchi Method indicated that pressure, mold temperature, melt temperature, and packing duration are of high significance in controlling the flatness of the components. Assembled device 1680 is resultant from the stacking of the components together. In embodiments, the removable cover 1620 is affixed onto the second base layer 1640, and then the combination therefrom is coupled onto the first base layer 1660 to form the assembled device 1680.

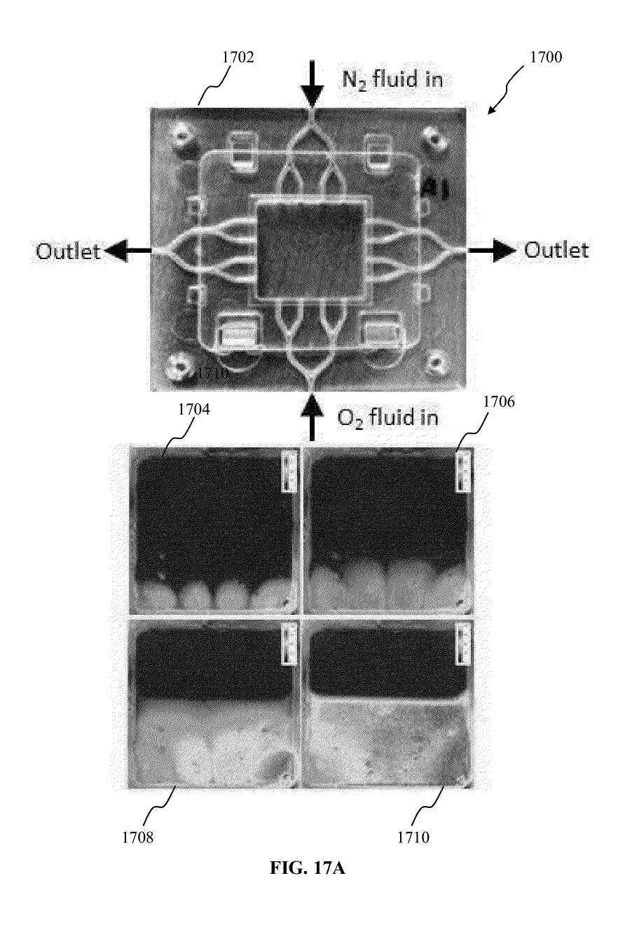

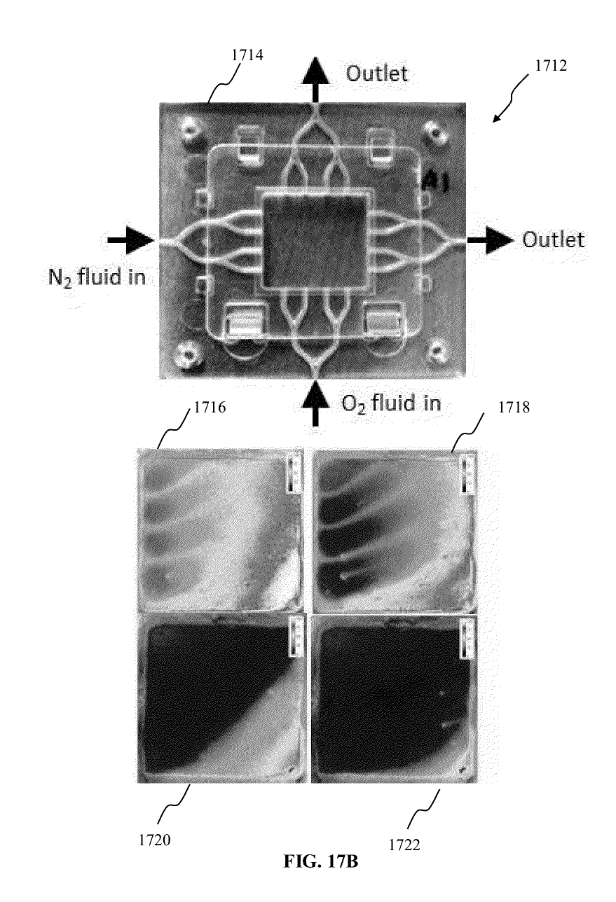

FIG. 17A and FIG. 17B illustrate mixing patterns of oxygen saturated water and nitrogen saturated water in the chamber when the two fluids enter and exit from different inlets. These mixing patterns represent the ability of the device to create reproducible chemical gradients ahead of inoculation with bacteria. FIG. 17A illustrates a first mixing pattern with oxygen and nitrogen saturated water and shows chemical gradients across a device when the two fluids enter the chamber from opposite sides (top and bottom) while the remaining sides (left and right) are configured as outlets. According to embodiments, the device is initially oxygen deficient due to the continuous flow of nitrogen saturated water from the top with respect to 1702 and the oxygen saturated fluid enters from the bottom. The glass-coverslip is coated with plant optrodes that enable the oxygen concentration to be mapped out in the plane of the cover-slip. Images 1704, 1706, 1708 and 1710 show the chemical gradient in the chamber, in a correspondingly sequential order. The amount of oxygen concentration in the chamber is shown to be increasingly saturated and attaining an equilibrium state, with respect to directional flow. Equilibrium is achieved in typically ten minutes or thereabouts.

FIG. 17B illustrates a second mixing pattern with oxygen and nitrogen saturated water. In FIG. 17B, plot 1712 shows time-lapsed photographs of the chemical gradients across the device when the two fluids enter the chamber from adjacent sides (left and bottom) and the remaining sides are configured as outlets. According to embodiments, the device and the setup for growth is initially saturated with oxygen since there is a continuous inflow of oxygen saturated fluid from bottom with respect to 114 and the nitrogen saturated water enters from the left. Images 1716, 1718, 1720, 1722 show the chemical gradient in the chamber, in a correspondingly sequential order. The amount of oxygen concentration in the chamber is shown to be increasingly dissipated, with respect to directional flow.

As one of the intentions of the device according to embodiments is for the investigation into the initial grown of biofilms, it may still necessary to only limit this growth to the cover slips or sample coupons. Depending on the bacterial type it is possible to delay the settlement on other surfaces such as the chamber walls and inlet and outlet channels. Geometry has a large part to play in this exercise. Sharp corners, rough surfaces, sections where the fluid stagnates will encourage the rapid settlement and growth of biofilms. Changing the chamber material has some effect as different polymers have different surface energies and biofilm growth is often sensitive to surface energy. Similarly, coatings can also be applied to change the surface from hydrophobic to hydrophilic targeting the preference of individual bacteria. However, the most significant reduction in attachment has been achieved through structuring of the surface or coating with materials with antimicrobial properties.

Various strategies can be applied to the device according to embodiments. Micro-embossing can be used to modify the surface topography once a device has been made by injection molding. Thin film coatings can also be made by mature coating technologies such as sputter deposition, in conjunction with a thin metal shadow mask, to limit the growth of the coating to the intended areas of interest.

Two model gram negative bacteria have been successfully grown in an embodiment of the device. These bacteria are pseudomonas aeruginosa and pseudomonas putida.

In various embodiments, there is provided a system for supporting growth of biological material. A system can include a device for supporting growth of biological material, as described above. Such a system can further include a data management system, the data management system including a processing unit a memory module. In embodiments, at least one computer program product is provided, the computer program product directly loadable into the memory module of the data management system of the system for supporting growth of biological material, the computer program product arranged to cooperate with various inputs in the system. According to embodiments, the data management system is coupled to the outputs of various sensors and monitoring equipment coupled or attached on to the device for detecting and monitoring growth of biofilm.

A computing system or a controller or a microcontroller or any other system providing a processing capability can be presented according to various embodiments in the present disclosure. Such a system can be taken to include a processor. The system according to various embodiments can include a controller which may include a memory which is for example used in the processing carried out by portions of the receiver. A memory used in the embodiments may be a volatile memory, for example a DRAM (Dynamic Random Access Memory) or a non-volatile memory, for example a PROM (Programmable Read Only Memory), an EPROM (Erasable PROM), EEPROM (Electrically Erasable PROM), or a flash memory, e.g., a floating gate memory, a charge trapping memory, an MRAM (Magnetoresistive Random Access Memory) or a PCRAM (Phase Change Random Access Memory).

In various embodiments, a “circuit” may be understood as any kind of a logic implementing entity, which may be special purpose circuitry or a processor executing software stored in a memory, firmware, or any combination thereof. Thus, in an embodiment, a “circuit” may be a hard-wired logic circuit or a programmable logic circuit such as a programmable processor, e.g. a microprocessor (e.g. a Complex Instruction Set Computer (CISC) processor or a Reduced Instruction Set Computer (RISC) processor). A “circuit” may also be a processor executing software, e.g. any kind of computer program, e.g. a computer program using a virtual machine code such as e.g. Java. Any other kind of implementation of the respective functions which will be described in more detail below may also be understood as a “circuit” in accordance with various alternative embodiments. Similarly, a “module” is thusly defined as a portion of a system according to various embodiments in the present disclosure and may encompass a “circuit” as above, or may be understood to be any kind of a logic-implementing entity therefrom.

According to the present disclosure, the following advantages are listed with regard to the device for supporting biological material growth according to embodiments:
The device is a disposable device.
The device can be sterilized by irradiation with gamma rays.
The device is a low unit cost afforded by use of use of mature manufacturing technology.
The device is a scaleable microfluidics platform from millimeters to meters.
The device can include a split design which allows sensors and additional materials to be sandwiched in between the top and bottom layers that constitute the device.
The device can include a non-metallic body.
The device can include single inlet and outlet channels that morph, in a binary manner, into quad channels entering into the chamber.
The device can include geometry designed to produce reproducible micro-hydraulics (velocity fields) in 3-dimensions.
The device can include reconfigurable as a multiple-well or array layout for anti-fouling agent screening.
The device can include monolithic integration of sensors within the platform.
The device can include an easy biofilm sampling option with the clip-on removable cover integrated with the microscope cover-slip.
The device can be optimized for use with high resolution confocal microscopes, which may have limited working distances.
The device can include a novel non-metal choice of antimicrobial solution to inhibit fouling of biofilm in the inlets/outlets of device.
The device can include reconfigurable inlet/outlet ports.
The device can include a unique inoculation port.
The device can provide for fluid tightness.
The device can provide for the interchangeability of device material.
The device can provide for bubble removal.

According to the present disclosure, the device for supporting biological material growth according to embodiments can be used in the following applications:
Rapid screening of antibiotics and anti-fouling agents.
Provision for real-time investigation of multi-species biofilms throughout their biological development;
Identification of unique ways to disperse bacteria before biofilm formation.
Identification of mixed species of bacteria necessary to degrade oil and other water and land born contaminants.

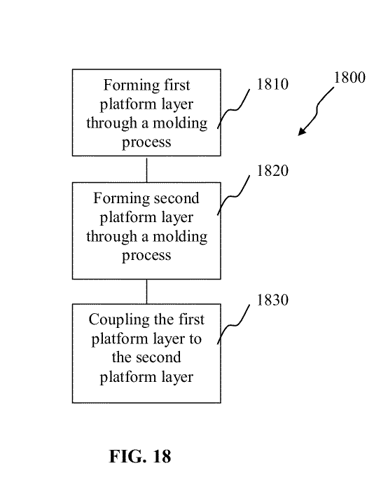

FIG. 18 illustrates a block schematic of a method of manufacturing a device according to an embodiment of the present disclosure. According to an embodiment, a method 1800 of manufacturing a device for supporting biological material growth is provided. In 1810, the method includes forming a first platform layer through a molding process. According to an embodiment, the first base layer includes a central chamber and a plurality of elongate channels coupling the central chamber to the periphery of the first platform layer. In 1820, the method includes forming a second platform layer through a molding process. In 1830, the method includes coupling the first platform layer to the second platform layer.

In an embodiment, the coupling of the first platform layer to the second platform layer is an airtight and permanent coupling.

In an embodiment, the coupling of the first platform layer to the second platform layer is any one of chemical welding, ultrasonic welding, laser welding, indium welding, or adhesion.

In an embodiment, the molding process is any one of injection molding, split-injection molding, powder injection molding, casting or machining.

In an embodiment, the first platform layer and the second platform layer include any one of thermoplastic, thermoset, copolymers, polycarbonate, flexi-glass, ceramic glass, or metallic materials.

In an embodiment, the method further includes coupling a removable cover to any one of the first platform layer and the second platform layer, the removable cover including a observation portion arranged for a viewing of the central chamber.

In an embodiment, the coupling of the removable cover is a snap-fit coupling.

In an embodiment, the method further includes utilizing a glass cover slip and a elastomeric gasket to provide the observation portion of the removable cover.

In an embodiment, the method further includes treating the channels for preventing biological material growth in the elongate channels, including any one of micro-embossing the elongate channels, coating the elongate channel with a ceramic overlay, coating the elongate channel with a polymer overlay, or coating the elongate channel with a metallic overlay.

In an embodiment, the method further includes sandwiching a metallic layer between the first platform layer and the second platform layer prior to coupling the first platform layer to the second platform layer.

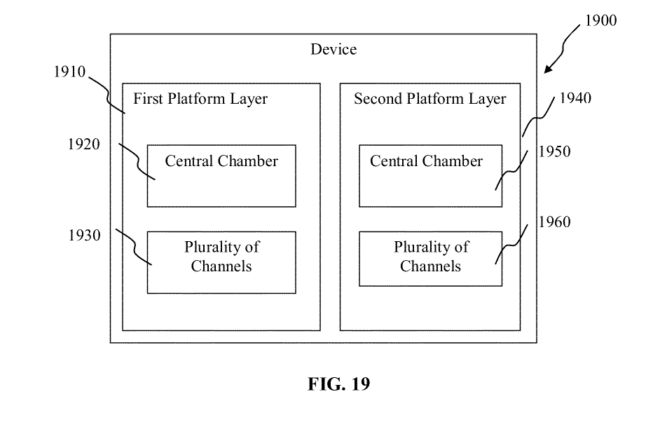

FIG. 19 illustrates a block schematic of a device according to an embodiment of the present disclosure. According to an embodiment, there is provided a device 1900 for supporting biological material growth. The device 1900 includes a first platform layer 1910, formed through a molding process, the first platform layer further including a central chamber 1920 and a plurality of elongate channels 1930 coupling the central chamber to the periphery of the first platform layer. The device 1900 further includes a second platform layer 1940, formed through a molding process. According to an embodiment, the first platform layer is coupled to the second platform layer to form a growth platform.

In an embodiment, the growth platform includes a plurality of peripheral apertures at the periphery of the growth platform, the peripheral apertures coupled to the central chamber through the elongate channels.

In an embodiment, the growth platform includes four peripheral apertures, each of the peripheral apertures provided perpendicular to each other.

In an embodiment, the device further includes a branch arrangement of channels between any one of the peripheral apertures and the central chamber.

In an embodiment, the branch arrangement of channels includes a channel leading from the peripheral aperture branching to form a plurality of channels leading to the central chamber.

In an embodiment, the channel leading from the peripheral aperture is branched twice to form the plurality of channels leading to the central chamber.

In an embodiment, the peripheral aperture is an extruded aperture.

In an embodiment, the device further includes a sensor within the device between the first platform layer and the second platform layer.

In an embodiment, the sensor is arranged to monitor growth of the biological material and is any one of a volumetric oxygen sensor, a plant optrode, an oxygen reporter, a thermocouple, magnetic fields sensors, high permeability structures, and electrical contact sensors.

In an embodiment, the device further includes an optical waveguide formed in the growth platform.

In an embodiment, the device further includes a Faraday cage structure formed in the device.

In an embodiment, the device further includes an inoculation port provided at a periphery of the growth platform.

In an embodiment, the inoculation port is coupled to the chamber by a inoculation port channel, the inoculation port channel separate from any fluid-carrying channel in the device.

The above apparatus, method and/or system as described and illustrated in the corresponding figures, is not intended to limit an or any apparatus, method or system as according to an embodiment, and the scope of the present disclosure. The description further includes, either explicitly or implicitly, various features and advantages of the method or system according to the present disclosure, which can be encompassed within an apparatus, method or system according to the disclosure.

While embodiments of the disclosure have been particularly shown and described with reference to specific embodiments, it should be understood by those skilled in the art that various changes in form and detail may be made therein without departing from the spirit and scope of the disclosure as defined by the appended claims. The scope of the disclosure is thus indicated by the appended claims and all changes which come within the meaning and range of equivalency of the claims are therefore intended to be embraced.

1. A method of manufacturing a device for supporting biological material growth, comprising:
forming a first platform layer through a molding process, the first platform layer comprising:
a central chamber; and
a plurality of elongate channels coupling the central chamber to the periphery of the first platform layer;
forming a second platform layer through a molding process; and
coupling the first platform layer to the second platform layer.

2. The method of claim 1, wherein the coupling of the first platform layer to the second platform layer is an airtight and permanent coupling.

3. The method of claim 2, wherein the coupling of the first platform layer to the second platform layer is any one of chemical welding, ultrasonic welding, laser welding, indium welding, or adhesion.

4. The method of claim 1, wherein the molding process is any one of injection molding, split-injection molding, powder injection molding, casting or machining.

5. The method of claim 1, wherein the first platform layer and the second platform layer comprise any one of thermoplastic, thermoset, copolymers, ceramic glass, or metallic materials.

6. The method of claim 1, further comprising:
coupling a removable cover to any one of the first platform layer and the second platform layer, the removable cover comprising an observation portion arranged for a viewing of the central chamber.

7. The method of claim 6, wherein the coupling of the removable cover is a snap-fit coupling.

8. The method of claim 6, further comprising:
utilizing a glass cover slip and a elastomeric gasket to provide the observation portion of the removable cover.

9. The method of claim 1, further comprising:
treating the channels for preventing biological material growth in the elongate channels, comprising any one of micro-embossing the elongate channels, coating the elongate channel with a ceramic overlay, coating the elongate channel with a polymer overlay, or coating the elongate channel with a metallic overlay.

10. The method of claim 1, further comprising sandwiching a metallic layer between the first platform layer and the second platform layer prior to coupling the first platform layer to the second platform layer.

11. A device for supporting biological material growth, comprising:
a first platform layer, formed through a molding process, the first platform layer comprising:
a central chamber; and
a plurality of elongate channels coupling the central chamber to the periphery of the first platform layer; and
a second platform layer, formed through a molding process;
wherein the first platform layer is coupled to the second platform layer to form a growth platform.

12. The device of claim 11, wherein the growth platform comprises a plurality of peripheral apertures at the periphery of the growth platform, the peripheral apertures coupled to the central chamber through the elongate channels.

13. The device of claim 12, wherein the growth platform comprises four peripheral apertures, each of the peripheral apertures provided perpendicular to each other.

14. The device of claim 13, further comprising a branch arrangement of channels between any one of the peripheral apertures and the central chamber.

15. The device of claim 14, wherein the branch arrangement of channels comprises a channel leading from the peripheral aperture branching to form a plurality of channels leading to the central chamber.

16. The device of claim 15, wherein the channel leading from the peripheral aperture is branched twice to form the plurality of channels leading to the central chamber.

17. The device of claim 11, wherein the peripheral aperture is an extruded aperture.

18. The device of claim 11, further comprising a sensor within the device between the first platform layer and the second platform layer.

19. The device of claim 18, wherein the sensor is arranged to monitor growth of the biological material and is any one of a volumetric oxygen sensor, a plant optrode, an oxygen reporter, a thermocouple, magnetic fields sensors, high permeability structures, and electrical contact sensors.

20. The device of claim 11, further comprising an optical waveguide formed in the growth platform.

21. The device of claim 11, further comprising a Faraday cage structure formed in the device.

22. The device of claim 11, further comprising an inoculation port provided at a periphery of the growth platform.

23. The device of claim 22, wherein the inoculation port is coupled to the chamber by an inoculation port channel, the inoculation port channel separate from any fluid-carrying channel in the device.
